(19) United States
(12) Patent Application Publication
(10) Pub. No.: US 2002/0119197 A1
(43) Pub. Date: Aug. 29, 2002

(54) Process and system for controlled-release drug delivery

(76) Inventors: Stephen Craig Dyar, Brighton, MI (US); Mebrahtu Fessehaie, Mineola, NY (US); Isaac Ghebre-Sellassie, Morris Plains, NJ (US); Monzer Michael Mayassi, Somerset, NJ (US); Matthew J. Mollan JR., Succasunna, NJ (US); Haimonot Woldegaber, Dover, NJ (US)

Correspondence Address: Charles W. Ashbrook, Warner-Lambert Company, 2800 Plymouth Road, Ann Arbor, MI, 48105, US

(21) Appl. No.: 10/007,177

(22) Filed: Dec. 5, 2001

Related U.S. Application Data
(60) Provisional application No. 60/251,996, filed on Dec. 7, 2000.

(51) Int. Cl. A61K 9/24

(52) U.S. Cl. 424/473

(57) ABSTRACT

A controlled-release dosage form for a pharmaceutically-active agent comprises a core, in which the agent is dispersed, surrounded by a diffusion-limiting sleeve. The agent is released at a zero-order or approximately linear rate because the release rate of the agent will be governed entirely by erosion from exposed core surfaces; the surface area of which does not change substantially during the release process. Such a product may be made by co-extruding the core and sleeve material and slicing the extrudate.

### FIELD OF THE INVENTION

The present invention relates to a co-extruded, controlled-release pharmaceutical dosage form and a method for manufacturing such a dosage form.

### BACKGROUND OF THE INVENTION

A common objective of convenience in medical treatment is to provide a pharmaceutically-active agent in a matrix and/or in a fully or partially surrounding coating which controls the release of the diffusion of the pharmaceutically-active agent into surrounding bodily fluid by chemical and/or mechanical means, thereby providing uniform dosing over a prolonged period following a single administration.

Among such prior proposals are those disclosed in the following patent publications:

U.S. Pat. No. 3,113,076 (Jacobs)

Jacobs describes a controlled-release medicament, in the form of a tablet, comprising “a substantially uniform mixture” of an active ingredient and a pharmaceutical vehicle. The tablet has at least one aperture to control the inner and outer surface areas of the medicament for controlling release characteristics. The tablet may be coated to further slow release of the active ingredient. The tablet is formed by pressing, molding, and/or extruding.

U.S. Pat. No. 4,755,180 (Ayer, et al)

Ayer, et al, describes a polymeric-coated osmotic dosage form with an exit hole (or other passageway) for direct release of an active ingredient from the interior of the polymer coating. The dosage form further comprises a semipermeable wall (polymeric coat impermeable to the active ingredient) surrounding a core of active ingredient combined with an “osmotically effective solute” which is said to control osmotic migration of fluid and active ingredient into and out of the semipermeable membrane enclosure.

U.S. Pat. No. 4,816,262 (McMullen)

McMullen describes a controlled-release tablet having a core of active drug substance coated by a hydrophobic material. A hole of varying dimension through the center of the tablet is said to provide a controllable mechanism for release of the active ingredient. The outer coating is applied to the tablet by dip coating.

U.S. Pat. No. 4,839,177 (Columbo et al)

Columbo et al describes a system for controlled-rate release comprising a “deposit-core” of a defined geometric shape containing an active ingredient adjacent to a water impermeable support platform partially coating the “deposit core” A swellable polymeric material, combined in the “deposit core” with the active ingredient, controls the intake of water by swelling. The coating does not allow release of the active ingredient. All of the active ingredient is released from the exposed portion of the “deposit core” upon swelling of the swellable polymer with which it is combined. The support platform is formed by compression of polymeric material, immersion in a solution of polymeric material, or by spraying the solution of polymeric material onto the core. U.S. Pat. No. 5,681,568 (Goldin et al)

Goldin et al describes a microporous membrane for delivery of macromolecules to a therapeutic target. The membrane contains a microporous underlayment, a microskin, and a macromolecule(s) in the form of a hollow fiber. Co-extrusion is used to form the hollow fiber. The fibrous material is extruded as the outer layer, and a polymer solution is extruded inside of the fibrous material such that a hollow, annular form is obtained.

European Patent Application Number 0 891 769 A1

This European patent application discloses a method in which a co-extrusion device is used to produce a controlled-release pharmaceutical dosage form. The outer extruded coating apparently completely surrounds a pharmaceutical agent-containing core.

Co-extrusion itself is also used in other applications, as shown, for example, in U.S. Pat. No. 5,417,992, which discloses the making of food products wherein a cheese filling is surrounded by a dough or puffed outer composition.

Notwithstanding the above-referenced prior information, the present inventors have developed what they believe to be a simpler and possibly more consistent controlled-release dosage form, as well as an improved method of making such a dosage form.

### SUMMARY OF THE INVENTION

In accordance with the present invention, a controlled-release drug dosage form comprises a central core, which includes a pharmaceutical agent, in a carrier matrix, typically a controlled-release composition. The core has two exposed opposite end surfaces and a peripheral surface at an outer edge of the core extending between the end surfaces. That peripheral surface is surrounded by a diffusion-limiting sleeve or coating. The diffusion-limiting sleeve is substantially impervious to water or bodily fluids, so that the release rate of the pharmaceutical agent in the central core is governed entirely by erosion or dissolution at the exposed surfaces. The surface area of each of the exposed faces remains substantially constant through the drug delivery period. Generally, the core and sleeve are cylindrical.

The present invention also comprises a method of making a controlled-release dosage form as a single or multiple unit dose by coextruding composite products comprising a central core of pharmaceutical agent of low or high water solubility, preferably in a matrix of a controlled-release composition, and a surrounding sleeve of a diffusion-limiting composition. The co-extrudate is sliced axially to produce a plurality of dosage forms each comprising a central core in which the pharmaceutical agent core has two oppositely faced exposed surfaces and is surrounded by the diffusion-limiting sleeve of outer co-extrudate. Preferably, the oppositely faced exposed surfaces are parallel to each other.

As used herein, controlled-release implies the controlled delivery of a drug substance at specific sites within the gastrointestinal tract of a mammal, and includes immediate, extended, pulsatile, enteric, and colonic release.

Controlled-release of other active ingredients and veterinary products may also be effected by incorporation of such active ingredients in a core and surrounding sleeve product analogous to that otherwise disclosed herein.

For a better understanding of the present invention, reference may be made to the following detailed description and the appended claims, taken together with the accompanying drawings.

### BRIEF DESCRIPTION OF THE DRAWINGS

FIG. 1 is a schematic view of a co-extrusion apparatus that can be used in the method of the present invention.

FIG. 2 is a cross-sectional view of the common die 17 of the co-extrusion apparatus shown in FIG. 1.

FIG. 3 is a perspective view of a pharmaceutical dosage form of the present invention.

FIGS. 4 and 5 are schematic views of barrel and screw designs for use in extruders useful in the method of the present invention.

FIG. 6 is a graphical representation of the linear release rate of a drug substance from an exemplary formulation made according to the present invention.

### DETAILED DESCRIPTION OF THE INVENTION

Soluble controlled-release dosage forms of pellets, disks, etc., by which pharmaceutical agents (or active ingredients such as pesticides or herbicides) are controllably released for dissolution to surrounding fluids are well-known. Typically, the pharmaceutically-active agent is disposed in a matrix material and/or coating material which controls the delivery of the pharmaceutically-active agent by controlling either the access of the surrounding bodily fluids to the pharmaceutically-active agent, or controls the release outwardly from the matrix material or coating of the pharmaceutically-active agent.

The present co-extruded controlled-release dosage form may utilize any pharmaceutically-active agent, preferably those active agents which are suitable for human consumption, and controlled-release matrix material compositions in which such agents (or other active ingredients) may be included. In addition, the agent or ingredient is embedded in an erodible matrix. Further, the present invention encompasses essentially any suitably active ingredient, which may or may not be water soluble, that can be formulated into a solid dosage form for desired release kinetics, such as zero-order controlled-release delivery. Examples of specific active ingredients which may be useful and, preferably, are suitable for human consumption, are listed below:

1. Antipyretic, analgesic and anti-inflammatory agents, such as indomethacin, aspirin, diclofenac sodium, ketoprofen, ibuprofen, mefenamic acid, dexamethasone, hydrocortisone, prednisolone, acetaminophen, phenylbutazone, flufenamic acid, sodium salicylate, tramadol hydochloride, oxaprozin, and etodolac.

2. Antiulcer agents, such as omeprazole, cimetidine, lansoprazole, ranitidine hydrochloride, famotidine, and nazatidine.

3. Coronary vasodilators, such as nifedipine, isosorbide dinitrate, diltizem hydrochloride, dipyridamole, isosorbide mononitrate, verapamil, nicardipine, nifedipine, and nitroglycerin.

4. Peripheral vasodilators, such as sildenafil citrate, cinepazide maleate, cyclandelate, and pentoxiphylline.

5. Antibiotics, such as ampicillin, amoxicillin, cefalexin, clarithromycin, cefuroxime axetil, cefropzil, erythromycin ethyl succinate, bacampicillin hydrochloride, minocycline hydrochloride, chloramphenicol, tetracycline, and erythromycin.

6. Synthetic antimicrobial agents, such as nalidixic acid, enoxacin, cinoxacin, levofloxacin, ofloxacin, norfloxacin, ciprofloxacin hydrochloride, and sulfamethoxazole-trimoethoprim.

7. Antispasmotic agents, such as propantheline bromide, atropine sulfate, and scopolamine.

8. Antitussive and antiasthmatic agents, such as theophylline, aminophylline, codeine phosphate, dextromethorphan, ephedrine hydrochloride, and noscapine.

9. Bronchodilators, such as salbutamol sulfate, pirbuterol hydrochloride, bitolterol mesilate, clenbuterol hydrochloride, terbutaline sulfate, mabuterol hydrochloride, fenoterol hydrobromide, and methoxyphenamine hydrochloride.

10. Diuretics, such as furosemide, acetazolamide, trichlormethiazide, cyclothiazide, hydrochlorothiazide, hydroflumethiazide, spironolactone, and triamterene.

11. Muscle relaxants, such as tolperisone hydrochloride, eperisone hydrochloride, tizanidine hydrochloride, mephenesin, chlorzoxazone, phenprobamate, methocarbamol, baclofen, and dantrolene sodium.

12. Cerebral metabolism improving agents, such as meclofenate hydrochloride.

13. Tranquilizers, such as oxazolam, diazepam, temazepam, meprobamate, nitrazepam, chlordiazepoxide, sulpiride, clocapramine hydrochloride, zotepine, chlorpromazine, and haloperidol.

14. Beta-Blockers, such as pindolol, propranolol hydrochloride, metoprolol tartrate, labetalol hydrochloride, oxprenolol hydrochloride, acebutolol hydrochloride, metoprolol succinate, bufetolol hydrochloride, alprenolol hydrochloride, and nadolol.

15. Antiarrhythmic agents, such as procainamide hydrochloride, disopyramide, quinidine sulfate, propafenone hydrochloride, and mexiletine hydrochloride. Antigout agents, such as allopurinol, probenecide, colchicine, warfarin sodium, and sulfinpyrazone.

16. Antigout agents, such as allopurinol, probenecide, colchicine, warfarin sodium, and sulfinpyrazone.

17. Anticoagulants, such as ticlopidine hydrochloride, dicoumarol, and warfarin potassium.

18. Antiepileptics, such as gabapentin, pregabalin, phenytoin, divalproex sodium, sodium valproate, and metharbital.

19. Antihistaminics, such as loratadine, cetirizine hydrochloride, chlorpheniramine maleate, fexofenade hydrochloride, celmastine fumarate, and cyproheptadine hydrochloride.

20. Antiemetics, such as difenidol hydrochloride, methoclopramide, and trimebutine maleate.

21. Antihypertensive agents, such as methyldopa, prazosin hydrochloride, bunazosin hydrochloride, clonidine hydrochloride, budralasine, bisporolol fumarate, hydrochlorothiazide, terazosin hydrochloride, and urapidil.

22. Sympathomimetric agents, such as dihydroergotamine mesilate, isoproterenol hydrochloride, and etilefrine hydrochloride.

23. Expectorants, such as bromhexine hydrochloride, carbocysteine and cysteine methyl ester hydrochloride

24. Oral antidiabetic agents, such as glubenclamide, glimepiride, gliprizide, metformin hydrochloride, troglitazone, rosiglitazone, pioglitazone, tolbutamide, and glymidine sodium.

25. Iron preparations, such as ferrous sulfate and dried iron sulfate.

26. Vitamins, such as vitamin B ₁₂, vitamin B₆, vitamin C, and folic acid.

27. Therapeutic agents for pollakiuria, such as flavoxate hydrochloride, oxybutynin hydrochloride, and terodiline hydrochloride.

28. Angiotensin converting enzyme inhibitors, such as enalapril maleate, enalaprilat, fosinopril sodium, alacepril, lisinopril, quinapril hydrochloride, ramipril, and delapril hydrochloride.

29. Other types of active ingredients, that can be formulated according to this invention include acetohexamide, ajamaline, alendronate sodium, amlodipine besylate, amylobarbitone, atorvastatin calcium, simvastatin, pravastatin, fluvastatin, rosuvastatin, bendrofluozide, benzbromarone, benzoate, benzylbenzoate, betametharzone, paroxetine hydrochloride, buproprion hydrochloride, buspirone hydrochloride, chloramphenicol, chloropropamide, chlorthalidone, clofibrate, conjugated estrogens, corticosteroids, diazepam, dicumerol, digitoxin, digoxin, dihydroxypropyltheophylline, diltiazem hydrochloride, doxazosin mesylate, ergot alkaloids, ethotion, felodipine, fluoxetine hydrochloride, fluconazole, fluvastatin sodium, frusemide, glutethimide, griseofulvin, hydrochlorothiazide, hydrocortisone, hydroflumethiazide, hydroquinone, hydroyalkylxanthines, indomethacin, isoxsuprine hydrochloride, ketoprofen, khellin, levothyroxine sodium, losartan potassium, lovastatin, meprobamate, nabilone, nefazodone hydrochloride, nicotinamide, nifedipine, nitrofuirantoin, novalgin, nystatin, papaverine, paracetamol, phenylbutazone, phenobarbitone, pravastin sodium, prednisolone, prednisone, primadonel, reserpine, risperidone, romglizone, salicylic acid, salmeterol xinafoatte, sertraline hydrochloride, simvastatin, spironolactone, sulphabenzamide, sulphadiamadine, sulphamethoxydiazine, sulphamerazine, succinylsulphathiazole, sulphamethizole, sulphamethoxazole, sulphathiazole, sulphisoxazole, sumatriptain succinate, testosterone, tolazoline, tolbutamide, trifluoperazine, trimethoprim, valsartan, zolpidem tartrate and other water insoluble or water soluble active ingredients.

Typical matrix materials with which the pharmaceutically-active agents are combined may be selected from polyethylene glycol 400, polyvinylalcohol, polymethacrylates, cellulose acetate phthalate, polyvinylpyrrolidone, hydroxypropylmethylcellulose phthalate, hydroxypropylmethylcellulose acetate succinate, and polysorbate 80. Hydroxypropylcellulose and hydroxypropylmethylcellulose may also be incorporated in such matrix materials as well as polyethylene glycol of various molecular weights, ranging from 400 or below to 8000 and above. The matrix material may also include other common excipients, additives and solubility-controlling compounds, etc.

In accordance with the present invention, a core, including a pharmaceutically-active agent in a controlled-release matrix material, typically includes two exposed parallel planar end surfaces, with the edges of the core extending between those surfaces surrounded by a coating which is essentially water impervious. Controlled-release of the pharmaceutically-active agent from a dosage form of this configuration occurs only at the substantially constant cross-sectional area of the exposed surfaces. Alternatively, the exposed surfaces may be slanted, curved or angled, or a combination thereof.

Suitable materials for the essentially water-impervious outer coating of the dosage form of this invention include polymethacrylate or ethylcellulose. Plasticizers, such as dibutylsebacate, glycerine, and tributylcitrate, may also be incorporated in the coating formulation.

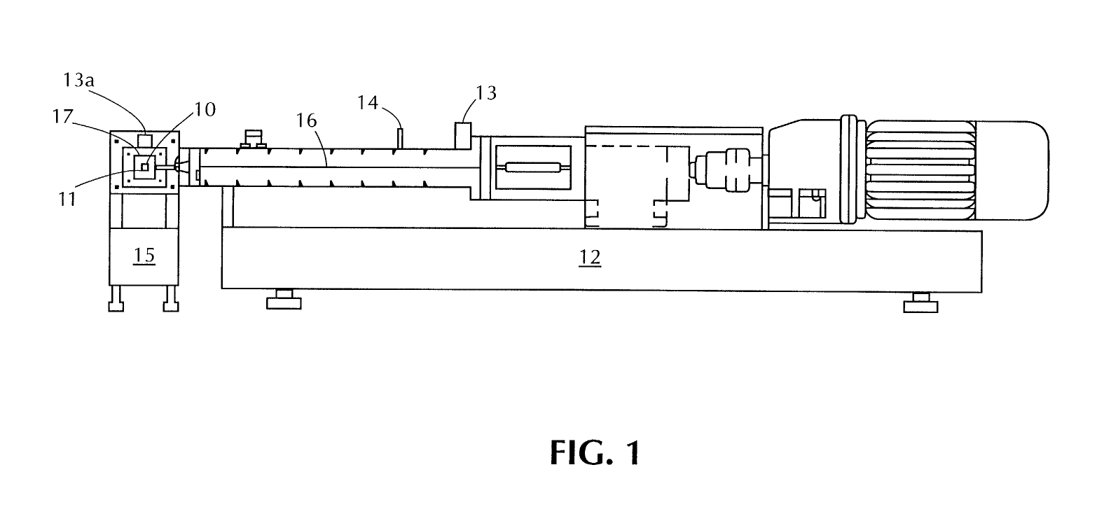

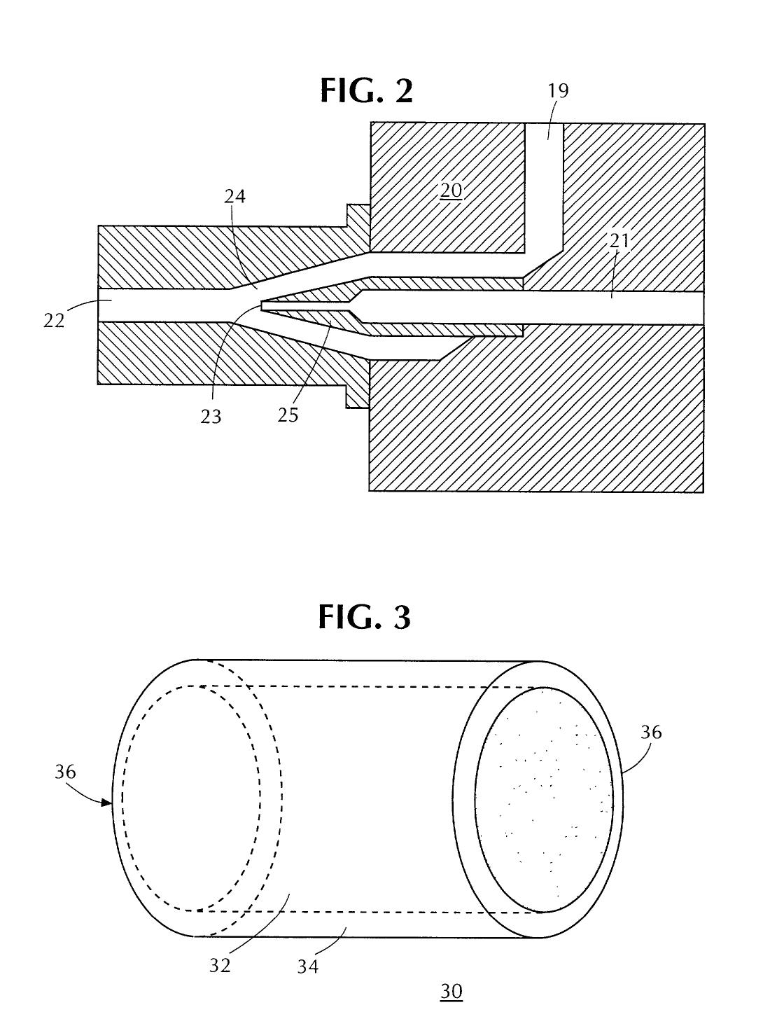

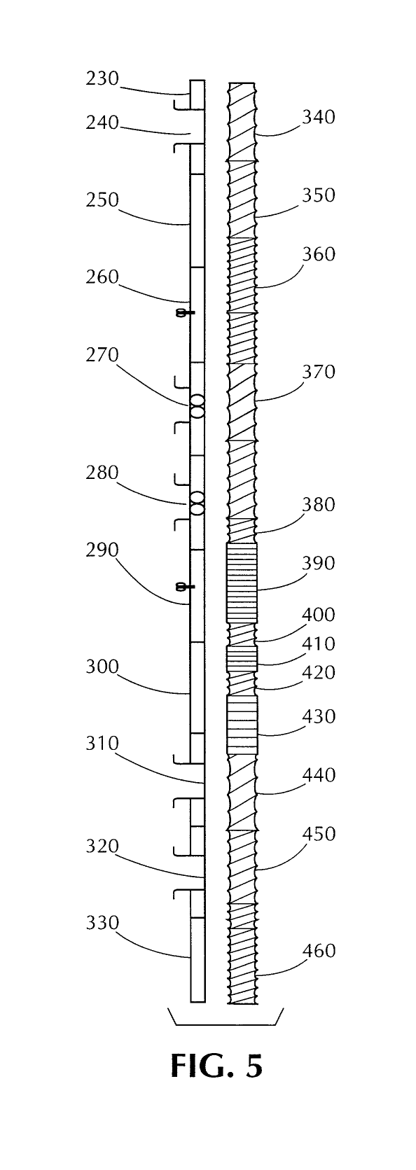

For the method of the present invention and for making the product of the present invention, an apparatus such as that shown schematically in FIGS. 1 and 2 may be used. This apparatus comprises a first extruder 12 which produces outer layer 11, and a second extruder 15 for producing inner core 10 of a co-extrudate product. The components for the outer layer, including the pharmaceutically-agent, are fed, for example, into first extruder 12 using appropriate powder and/or liquid feed ports (13 and 14, respectively), while the components for the inner core are fed into second extruder 15 through appropriate powder-feed ports 13a and/or liquid-feed ports (not shown). Each extruder may be a twin-screw extruder having screws 16 (shown schematically) rotating in the same or opposite direction. Examples of such twin-screw extruder screws are described below with reference to FIG. 4 and 5. To melt or soften the materials being mixed in and extruded from extruders 12 and 15, heating elements (not shown) may be provided with sufficient capacity to melt or soften materials in the extruders, for example, to a temperature in the range of 30 C. to 250 C., preferably 40° C. to 200° C.

As shown in FIG. 2, which is a bottom cross-sectional view of the mixing dies at the outlets of extruders 12 and 15, the outlet passage 19 of first extruder 12, and outlet passage 21 of second extruder 15, intersect at a 90° angle in co-extrusion die 20, which in turn leads downstream to central core outlet 23, and a surrounding sleeve outlet 24, and from there to die outlet 22, from which is extruded the co-extrudate controlled-release drug delivery product. Surrounding sleeve outlet 24 extends entirely around the periphery of internal guide 25.

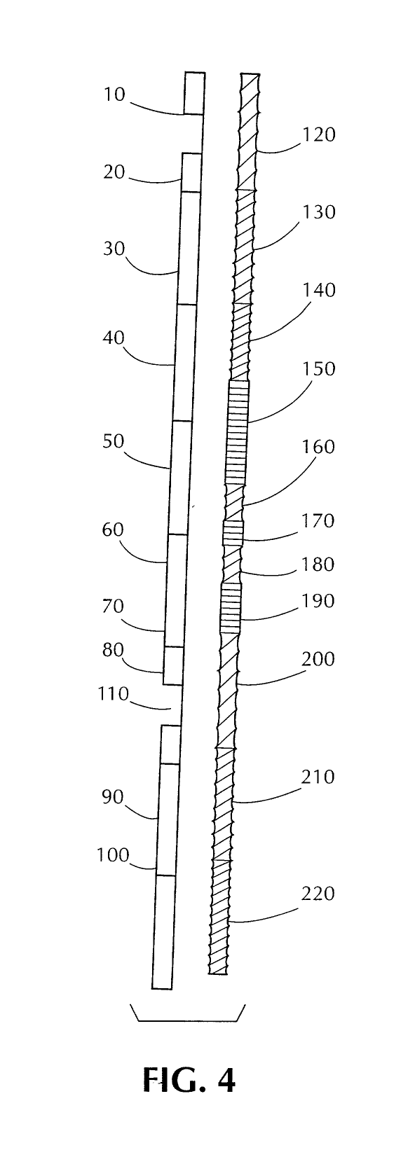

The co-extrudate dosage form of the present invention may be sliced along diametrical planes to form a controlled-release tablet or pellet, such as the cylindrical pellet 30 shown in FIG. 3. This slicing can occur either before or after hardening of the co-extrudate produced in the apparatus as shown in FIGS. 1 and 2. Typically, the slicing is accomplished by mechanical slicing or by laser slicing. This dosage form includes central core 32 containing the pharmaceutically-active agent, surrounded by an essentially water or bodily fluid impermeable outer coating sleeve 34. Core 32, at the ends of cylindrical pellet 30, includes two parallel oppositely planar exposed end surfaces 36, from which the pharmaceutically-active agent may be dissolved, eroded, diffused, or otherwise delivered substantially uniformly to the mammal to which it is administered. When the outer sleeve 34 is impermeable and nonerodible, at least within the time frame of drug release, exposed surfaces 36 provide a constant surface for drug release (i.e., the surface area from which the drug is released remains constant throughout the drug release period).

Controlled release cylindrical pellets can be introduced into a capsule, such as a gelatin capsule, soft gel capsule, hard gel capsule, and the like.

A pelleted dosage form, preferably having a total length less than the sum of the two diameters, more preferably one diameter of the pelleted dosage form, as shown in FIG. 3, may be produced from the following exemplary materials, as shown in Table 2 and Table 2.

TABLE 1
Composition of Exemplary Coating Material

| | Polymer | | Plasticizer | |
|---|---|---|---|---|
| Function | Name | Amount (%) | Name | Amount (%) |
| Coat | Eudragit RS | 95.0 | Triethylcitrate | 5.0 |
| Coat | Cellulose | 95.0 | Triethylcitrate | 5.0 |
| | Acetate | | | |
| Coat | Ethylcellulose | 95.0 | Triethylcitrate | 5.0 |
| Coat | Cellulose | 80.0 | Triethylcitrate | 20.0 |
| | Acetate | | | |

TABLE 2
Composition of Exemplary Core Material

| | Polymer | | Plasticizer | | API | |
|---|---|---|---|---|---|---|
| | | Amount | | Amount | | Amount |
| Function | Name | (%) | Name | (%) | Name | (%) |
| Core | PVP | 60.0 | PEG 400 | 10.0 | Sodium | 30 |
| | | | | | Phenytoin | |
| Core | PVP | 43.5 | PEG 400 | 10.0 | Troglitazone | 43.5^{a} |

API = Active pharmaceutical ingredient.
^{a}The last sample also includes Tween 80 at 3.0%.

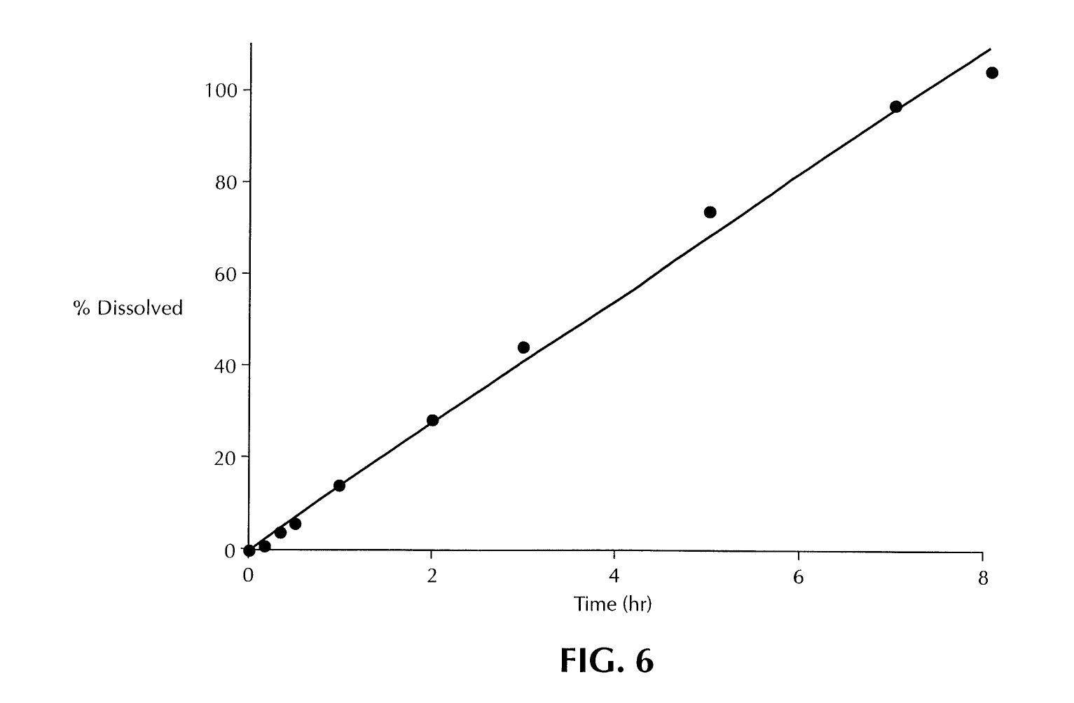

Pellets made in accordance with this invention comprise a core composed of a glassy matrix in which the pharmaceutically-active ingredient (API) (such as those set forth in Table 2 is dispersed molecularly or as particulate within a coating material, such as those set forth in Table 1. Because the core is typically composed of a glassy matrix, water and other fluids do not penetrate deeply into the exposed surfaces of the core. Therefore, the shape of the exposed core surfaces are substantially maintained during the entire course of drug release (as is the surface area, as described above), thereby providing an approximately linear release profile, as shown in FIG. 6. Preferably, the release profile remains approximately linear until about 80% of total amount of API in the core has been released.

It will be recognized by those skilled in the art that a wide range of other materials may also be used as one or both of the core and outer sleeve materials in the co-extrudate product, and in the sliced co-extruded controlled-release dosage form produced therefrom. It will also be appreciated that the method of this invention may be implemented by those skilled in the art with a wide range of equipment and process parameter variables, all tailored to the production of the specific co-extrudate product. The preparation of co-extrudate controlled-release dosage forms is further illustrated by the following working examples. The examples are illustrative only, and are not to be construed as limiting the invention in any respect.

### EXAMPLE 1

Using the materials listed in Table 1, a formulation of troglitazone in PVP was prepared using a Leistritz Micro 18/GL 40D twin-screw extruder (Leistriz Inc, Somerville, N.J.) with the screw design shown in FIG. 4 (showing one barrel and one screw from a set composed of two barrels and two screws) to extrude the outer layer. The coating polymer material was introduced into the extruder via a K-Tron gravimetric feeder, and the coating plasticizer was introduced via a Masterflex peristaltic pump. The fed materials were mixed and heated as they were processed progressively through barrel segments 20, 30, 40, 50, 60, 70, 80, 90, and 100 by screw segments 120, 130, 140, 150, 160, 170, 180, 190, 200, 210, and 220 located within the barrel. The length of these screw segments was 90, 90, 60, 30, 30, 20, 30, 20, 90, 90, and 90 mm, respectively, and the internal diameters of the two barrels were 18 millimeters. The heater set point and processing temperature for zones 10-50, as shown in the left part of FIG. 4, was 150° C., and was 145° C. for zones 60-100. An injection port (not shown) is located in barrel segment 40 over screw segment 140. A vent port 110 is located in barrel segment 80. The Eudragit RS (polyethyl acrylate, methyl methacrylate, trimethylammonioethyl methacrylate chloride, in a 1:2:0.1 ratio) powder feed rate was 2.72 kg/h, while the liquid triethylcitrate (TEC) feed rate was 2.72 mL/min.

A Leistritz Micro 27/GL 40D twin-screw extruder with the screw design as shown in FIG. 5 was used to extrude the core (comprised of materials such as those shown in Table 2). The materials (troglitazone/PVP) were introduced into the extruder via a K-Tron twin-screw feeder at port 240, and the PEG 400/Tween 80 mixture was introduced via a Masterflex peristaltic pump via injection port located in barrel segment 260. As the feed materials were mixed, they traversed the screws of FIG. 5, after being fed through main feed throat 240 located in barrel segment 230, through barrel segments 230, 250, 260, 270, 280, 290, 300, 310, 320, and 330, with inside barrel diameters of 27 mm, and screw segments 340, 350, 360, 370, 380, 390, 400, 410, 420, 430, 440, 450 and 460. The length of the screw segments was 90, 90, 150, 180, 30, 30, 30, 30, 30, 10, 90, 90 and 120 mm, respectively. Injection ports were located in barrel segments 260 and 290 over screw segments 360 and 390, respectively. In this example, nothing was added via the injection port located in barrel segment 290. Vent ports were located in barrel segments 310 and 320. In this apparatus, additional injection feed ports may be located in barrel segments 270 and 280. The temperature control set point and processing temperature for zones 230 and 250-280 was 150° C., and for zones 290-330 it was 145° C. Troglitazone was mixed uniformly with PVP polymer, and the troglitazone/PVP powder feed rate was 9.53 kg/h, while the liquid PEG 400/Tween 80 mixture feed rate was 24 mL/minute.

The extrudates of these respective extruders (the core and the coating) were combined in a common die with a 9:1 ratio (core:coat) which connected the two extruders at a 90° angle as shown in FIG. 2. Other angular alignments, such as 180° , are also possible. A Domier fan-cooled (cooling) tunnel (Dornier Manufacturing, Inc, Hartland, Wis.) was used to cool the co-extrudate material. The cooled co-extrudate was pelletized as described above using a Conair pelletizer (Conair Inc, Pittsburgh, Pa.) to produce pellets as shown in FIG. 3.

The twin screws in the extruders of FIGS. 4 and 5 generally are operated at 314 and 264 rpm, respectively. These screw speeds may be varied, however, within a wide range, for example, from about 10 to about 1500 rpm, but preferably between 100 and 400 rpm.

In one exemplary composition, a solid dispersion composed of 42.5% troglitazone, 3.5% Tween 80, and 11.5% PEG 400 with an ethylcellulose outer coating layer was tested. The pelletized sample was 12 mm in length and 7 mm in diameter. Dissolution studies were carried out in 900 mL of dissolution medium (0.1 M phosphate buffer with 0.5% SLS, pH 8) maintained at 37° C. using a USP dissolution apparatus II at 75 rpm. After introducing the sample into the dissolution medium, the solution was periodically sampled (10 mL aliquots) over 8 hours. The test samples were filtered through a Gelman Acrodisc® 0.45μm filter pad (Pall Life Sciences, Ann Arbor, Mich.), and the filtrate was collected after discarding the first 2-mL portion. The samples were diluted by a factor of two (v/v) with methanol prior to testing. The extent of drug dissolved in the dissolution medium was determined using an HP 8453 diode array spectrophotometer at 284 nm.

The troglitazone core ethylcellulose coated samples were obtained by slicing portions from a long strand. The samples were coated, as described above, on all sides except on the cross-sectional faces. The integrity of the coating was maintained during the dissolution run. This sample was tested for percent dissolved and active ingredient (API) release rate. The core material appeared visually to erode and to dissolve from the cross-sectional exposed surface core. A linear dissolution profile (R=0.99447) was obtained from this sample, indicating that an 8-hour, zero-order controlled-release profile was achieved with 100% release. This is graphically illustrated in FIG. 6.

### EXAMPLE 2

By following the general procedure set forth in Example 1, a pellet comprising an agent useful for treating Alzheimer's disease was prepared. The API was the compound 1-aza-bicyclo[2.2.1]heptan-3-one-[3-(3-methoxyphenyl)-prop-2-ynyl]-oxime, also referred to as CI-1017 (see U.S. Pat. No. 5,346,911). Pellets having two different concentrations of components were prepared, as follows:

| API (% w/w) | PVP (% w/w) | PEG 400 (% w/w) |
|---|---|---|
| 17.6 | 70.4 | 12.0 |
| 72.8 | 18.2 | 9.0 |

In both cases, the twin-screw extruders for both the core and the coating were operated at 150 rpm. The rate of co-extrusion in both cases was 10 pounds per hour. The polyvinylpyrrolidone K-30 (PVP) and the API (CI-1017) were blended to uniformity for 20 minutes in a 16-quart V-blender. The coating rate was held constant at 1 pound per hour of Eudragit RSPO, containing 5% (w/w) triethylcitrate (TEC). The coating mixture was extruded using a melt temperature between about 157° C. to about 161° C. The CI-1017 core was extruded at 100° C., and a melt pressure of 260 psi when the PEG level was 12% (w/w). A slightly lower pressure was used for the 9.0% level of PEG. The pellets of each concentration were uniform and dissolved uniformly over prolonged periods.

### EXAMPLE 3

By following the general procedures of Examples 1 and 2, the following co-extruded dosage forms of CI-1011 were prepared:

| Ingredients | | | Extrusion Rate |
|---|---|---|---|
| API (% w/w) | PVP (% w/w) | PEG 400 (% w/w) | (lbs/hr) |
| 17.0 | 68.1 | 14.9 | 5 |
| 17.3 | 69.1 | 13.6 | 7 |
| 17.3 | 69.1 | 13.6 | 10 |

CI-1011 is an agent useful for regulating lipids in mammals. It is now known generically as Avasimibe, and chemically is [(2,4,6-triisopropyl-phenyl)-acetyl]-sulfanic acid 2,6-diisopropyl phenyl ester (see U.S. Pat. No. 5,491,172). The pellets prepared with CI-1011 displayed uniform dissolution over prolonged periods.

### EXAMPLE 4

A formulation of Cl-1027 was prepared according to the methods of Examples 1-3. Cl-1027 is an agent useful for treating diabetes and for elevating HDL-cholesterol in mammals, and chemically is 6,6′-oxybis[2,2-dimethyl]-hexanoic acid (see U.S. Pat. No. 5,783,600). The core for the CI-1027 blend was extruded at a melt temperature of 195° C., with a melt pressure of 1200 psi. A vacuum was applied to the vent (in barrel segment 310 in FIG. 5) to reduce the number of bubbles that formed during the extrusion processing. The coating mixture was co-extruded, and the extrudate was sliced into pellets. The pellets had a composition of 17.6% (w/w) of CI-1027, 70.4% (w/w) PVP, and 12.0% (w/w) PEG 400. The co-extrusion was conducted so as to produce 10 pounds of extrudate per hour.

While this invention has been described with respect to specific embodiments thereof it is not limited thereto. Nor is the present invention limited to the example shown. Rather, various modifications may be made in the details of the invention and its various embodiments as described and exemplified without departing from the true spirit and scope of the invention. The claims which follow should be construed accordingly.

What is claimed is:

1. Pharmaceutical dosage form comprising a central core including a pharmaceutical agent in a controlled-release composition, said core having two exposed opposite end surfaces and a peripheral surface at an outer edge of said core extending between said two opposed end surfaces, said peripheral edge surrounded by a diffusion-limiting sleeve, wherein said sleeve limits the diffusion of fluids into said core.

2. Pharmaceutical dosage form, as recited in claim 1, wherein said core and said sleeve are cylindrical and said opposite surfaces are circular.

3. Pharmaceutical dosage form, as recited in claim 2, wherein the length of said sleeve is less than the diameter of said sleeve.

4. Pharmaceutical dosage form, as recited in claim 1, wherein said pharmaceutical agent comprises an agent selected from troglitazone, rosiglitazone, or proglitazone.

5. Pharmaceutical dosage form, as recited in claim 1, wherein said diffusion-limiting sleeve material comprises at least one of ethylcellulose and polymethacrylate (Eudragit RS).

6. Pharmaceutical dosage form, as recited in claim 5, further comprising a plasticizer.

7. Pharmaceutical dosage form, as recited in claim 6, wherein said plasticizer comprises at least one of triethylcitrate, dibutylsebacate, glycerin, and tributylcitrate.

8. Pharmaceutical dosage form, as recited in claim 1, wherein said core further comprises a matrix material.

9. Pharmaceutical dosage form, as recited in claim 8, wherein said matrix material comprises at least one material selected from the group consisting of polyethylene glycol, polyvinylalcohol, polymethacrylate, cellulose acetate phthalate, polyvinylpyrrolidone, hydroxypropylcellulose phthalate, hydroxypropylmethylcellulose, hydroxypropylmethylcellulose acetate succinate, hydroxypropylcellulose, hydroxypropyethylcellulose, and polysorbate 80.

10. Pharmaceutical dosage form, as recited in claim 9, wherein said matrix material comprises polyvinylpyrrolidone and polyethylene glycol.

11. Pharmaceutical dosage form, as recited in claim 10, wherein said polyethylene glycol comprises polyethylene glycol having a molecular weight ranging from 400 to 8000.

12. Pharmaceutical dosage form, as recited in claim 1, wherein said opposite surfaces are planar and are parallel to each other.

13. Pharmaceutical dosage form, as recited in claim 1, wherein said pharmaceutical agent comprises troglitazone and wherein said core further comprises a matrix material comprising polyvinylpyrrolidone and polyethylene glycol 400.

14. Pharmaceutical dosage form, as recited in claim 13, wherein said diffusion-limiting sleeve material comprises a material selected from the group consisting of at least one of ethylcellulose and polymethacrylate.

15. Pharmaceutical dosage form, as recited in claim 14, further comprising a plasticizer.

16. Pharmaceutical dosage form, as recited in claim 15, wherein said plasticizer comprises at least one of triethylcitrate, dibutylsebacate, glycerine, and tributylcitrate.

17. Method of making a pharmaceutical dosage form comprising coextruding an indefinite length of a central core including a pharmaceutical agent disposed in a controlled-release composition, surrounded by a diffusion-limiting composition, and slicing said co-extrudate across the longitudinal axis thereof.

18. Method of making a pharmaceutical dosage form, as recited in claim 17, wherein said co-extrudate is permitted to harden before said co-extrudate is sliced across the longitudinal axis thereof.

19. Method of making a pharmaceutical dosage form, as recited in claim 17, wherein said co-extrudate is sliced across the longitudinal axis thereof in parallel, perpendicular to said longitudinal axis.

20. Method of making a pharmaceutical dosage form, as recited in claim 17, wherein said co-extrudate is heated to a temperature in the range of 30° C. to 250° C.

21. Method of making a pharmaceutical dosage form, as recited in claim 20, wherein said co-extrudate is heated to a temperature in the range of 40° C. to 200° C.

22. Pharmaceutical dosage form, as recited in claim 1, wherein said composition is introduced into a capsule.

23. Method of making a pharmaceutical dosage form, as recited in claim 19, wherein said co-extrudate is sliced with a laser across the longitudinal axis of the co-extrudate in parallel, perpendicular to said longitudinal axis.

24. Pharmaceutical dosage form, as recited in claim 1, wherein the core of said composition is released by erosion.
